(19) United States
(12) Patent Application Publication
(10) Pub. No.: US 2023/0043585 A1
(43) Pub. Date: Feb. 9, 2023

(54) ULTRASOUND DEVICES FOR MAKING EYE MEASUREMENTS

(71) Applicant: META PLATFORMS TECHNOLOGIES, LLC, Menlo Park, CA (US)

(72) Inventors: Andre G. Golard, Seattle, WA (US); Sachin Talathi, Snoqualmie, WA (US)

(21) Appl. No.: 17/396,628

(22) Filed: Aug. 6, 2021

(51) Int. Cl. G02B 27/00; A61B 8/10; G01S 15/06; G01S 15/88; G02B 27/01

(52) U.S. Cl. CPC G02B 27/0093; A61B 8/10; G01S 15/06; G01S 15/88; G02B 27/0172; A61B 3/111

(57) ABSTRACT

The disclosed ultrasound devices may include at least one ultrasound transmitter positioned and configured to transmit ultrasound signals toward a user's face to reflect off a facial feature of the user's face and at least one ultrasound receiver positioned and configured to receive and detect the ultrasound signals reflected off the facial feature. At least one processor may be configured to receive data from the at least one ultrasound receiver and to determine, based on the received data from the at least one ultrasound receiver, at least one of the following eye measurements: an interpupillary distance of the user; an eye relief; or a position of a head-mounted display relative to the facial feature of the user. Various other devices, systems, and methods are also disclosed.

### BRIEF DESCRIPTION OF THE DRAWINGS

The accompanying drawings illustrate a number of example embodiments and are a part of the specification. Together with the following description, these drawings demonstrate and explain various principles of the present disclosure.

FIGS. 1A-1C illustrate various eye measurements that may be made by systems and devices of the present disclosure.

FIG. 2 is a front view of an eyeglass device illustrated over an eye of a user, according to at least one embodiment of the present application.

FIGS. 3A-3C are front views of an eyeglass device illustrated at three different facial positions, respectively, according to at least one embodiment of the present application.

FIG. 4 is a block diagram of an ultrasound system for making eye measurements, according to at least one embodiment of the present application.

FIG. 5 is a flow diagram of a method for making eye measurements, according to at least one embodiment of the present application.

FIG. 6 is an illustration of example augmented-reality glasses that may be used in connection with embodiments of this disclosure.

FIG. 7 is an illustration of an example virtual-reality headset that may be used in connection with embodiments of this disclosure.

Throughout the drawings, identical reference characters and descriptions indicate similar, but not necessarily identical, elements. While the example embodiments described herein are susceptible to various modifications and alternative forms, specific embodiments have been shown by way of example in the drawings and will be described in detail herein. However, the example embodiments described herein are not intended to be limited to the particular forms disclosed. Rather, the present disclosure covers all modifications, equivalents, and alternatives falling within the scope of the appended claims.

### DETAILED DESCRIPTION OF EXAMPLE EMBODIMENTS

Various eye measurements can be useful for ophthalmology, optometry, and head-mounted display systems. For example, interpupillary distance (IPD) is a measurement of the distance between a user's pupils. Ophthalmologists and optometrists may use IPD to customize eyeglass lens placement for the user by locating focal centers of eyeglass lenses at the user's IPD, which may improve the user's vision through the eyeglass lenses. The neutral position of the eyeglasses on the user's face may also be considered when locating the focal centers of the eyeglass lenses on an eyeglasses frame.

Head-mounted display (HMD) systems may include a near-eye display (NED) to display artificial-reality content to a user. Artificial reality includes virtual reality, augmented reality, and mixed reality. These systems often include lenses between the user's eye and the NED to enable the displayed content to be in focus for the user. The user's visual experience may be improved by aligning the user's pupils with focal centers of the lenses on the HMD system. Some conventional virtual-reality HMDs include a manually operated slider or knob for the user to adjust an IPD setting to match the user's IPD to more clearly view the content displayed on the NED. The user may also need to manually adjust a position of the HMD on the user's face to clearly view the displayed content.

A distance between the user's eye and a lens or NED of an HMD system, which is referred to as eye relief, may affect whether the user views the displayed content clearly and without discomfort. With traditional HMD systems, the eye relief may be manually adjusted by shifting the HMD on the user's head or by inserting a spacer between the HMD and the user's face. As the user's head moves, the HMD system may shift on the user's face, which may change the eye relief over time.

Eye tracking can also be useful for HMD systems. For example, by identifying where the user is gazing, some HMD systems may be able to determine that the user is looking at a particular displayed object, in a particular direction, or at a particular optical depth. The content displayed on the NED can be modified and improved based on eye-tracking data. For example, foveated rendering refers to the process of presenting portions of the displayed content in focus where the user gazes, while blurring (and/or not fully rendering) content away from the user's gaze. This technique mimics a person's view of the real world to add comfort to HMD systems, and may also reduce computational requirements for displaying the content. Foveated rendering may require information about where the user is looking to function properly.

Eye tracking is conventionally accomplished by directing one or more optical cameras at the user's eyes and performing image analysis to determine where the user's pupil, sclera, iris, and/or cornea is located. The optical cameras may operate at visible light wavelengths or infrared light wavelengths. The camera operation and image analysis often require significant electrical power and processing resources, which may add expense, complexity, and weight to HMDs. Weight can be an important factor in the comfort of HMDs, which are usually worn on the user's head and against the user's face.

The present disclosure is generally directed to using ultrasound for making eye measurements including, for example, IPD, eye relief, and glasses position. As will be explained in greater detail below, embodiments of the present disclosure may include ultrasound devices including at least one ultrasound transmitter and at least one ultrasound receiver for making such eye measurements. The ultrasound transmitter and ultrasound receiver may be implemented separately in different locations, or as ultrasound transceiver that both transmits an ultrasound signal and receives the reflected ultrasound signal. These ultrasound devices may be used as a standalone device or in connection with another sensor (e.g., ultrasound sensors configured for eye tracking) for calibration purposes. In some examples, machine learning may be employed to facilitate making the eye measurements based on data from the at least one ultrasound receiver. Embodiments of this disclosure may have several advantages over traditional systems that may employ only optical sensors. For example, ultrasound devices may be less expensive and bulky and may have less processing and power requirements than conventional systems that use only optical sensors for sensing eye measurements.

Features from any of the embodiments described herein may be used in combination with one another in accordance with the general principles described herein. These and other embodiments, features, and advantages will be more fully understood upon reading the following detailed description in conjunction with the accompanying drawings and claims.

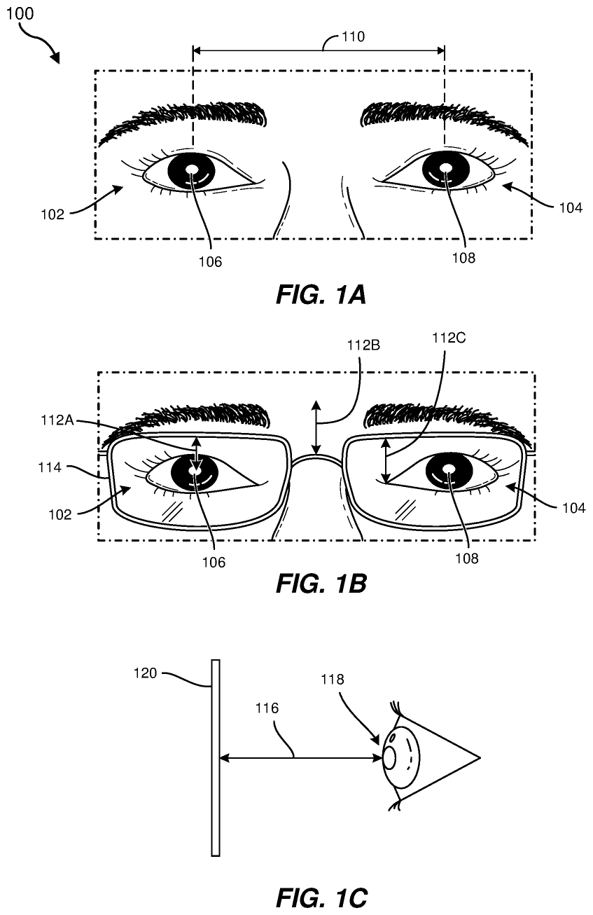

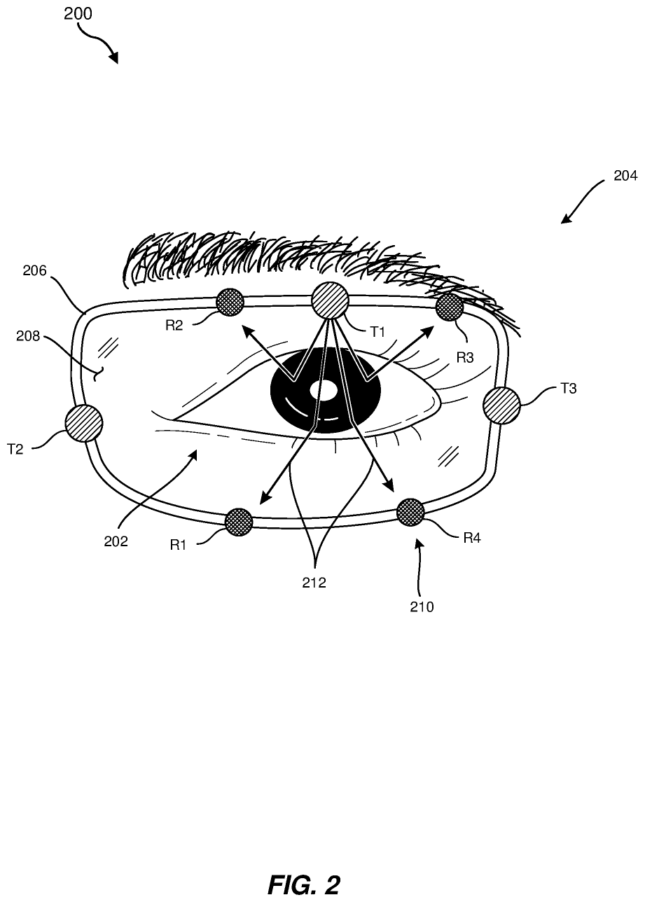

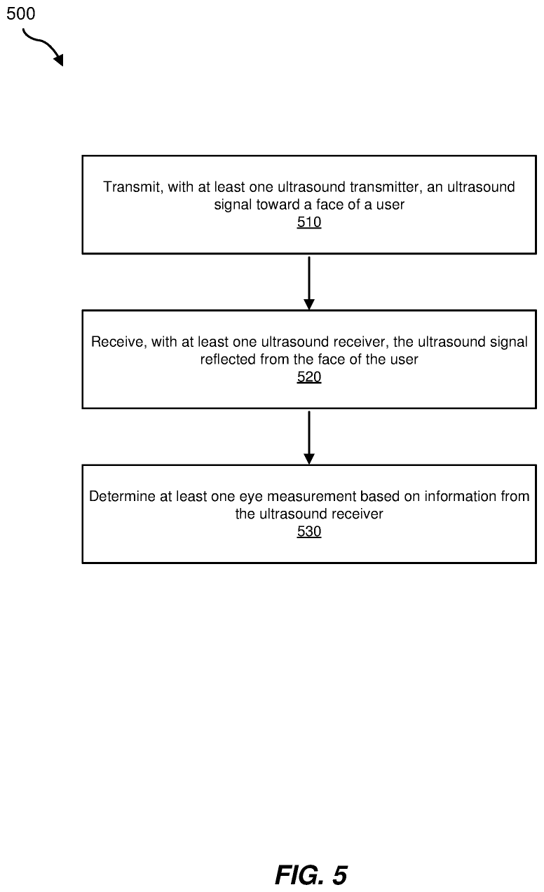

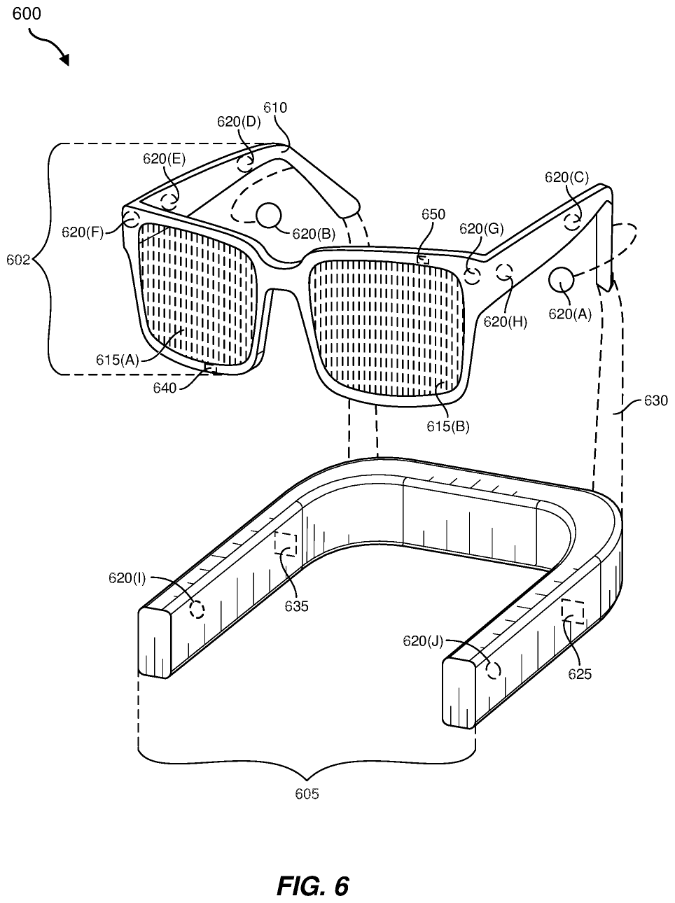

The following will provide, with reference to FIGS. 1A-1C, detailed descriptions of various eye measurements that may be made with ultrasound devices and systems of the present disclosure. With reference to FIGS. 2-4, descriptions of ultrasound devices and systems for making eye measurements will be provided. Detailed descriptions of methods of making eye measurements will follow with reference to FIG. 5. With reference to FIGS. 6-9, the following will provide detailed descriptions of example systems and devices that may employ concepts of the present disclosure.

FIGS. 1A-1C illustrate various eye measurements that may be made by systems and devices of the present disclosure. A portion of a face of a user 100 is shown, including the user's right eye 102 and the user's left eye 104. The right eye 102 may include a right pupil 106 and the left eye 104 may include a left pupil 108.

Referring to FIG. 1A, an interpupillary distance (IPD) 110 may be defined as a distance between the right pupil 106 and 108, typically measured in millimeters. The IPD 110 of various users 100 may be different. In addition, the IPD 110 may change depending on the vergence of the eyes 102, 104. The vergence of the eyes 102, 104 changes when the user 100 focuses on objects at different distances from the eyes 102, 104. For example, the IPD 110 will be less when the user 100 looks at a close object compared to looking at a far object, as the eyes 102, 104 turn inward to gaze at the close object.

Referring to FIG. 1B, various glasses positions 112A, 112B, 112C (collectively referred to as glasses position(s) 112) of eyeglass frames 114 are illustrated. The glasses position(s) 112 may be measured between a point or points on the eyeglass frames 114 and a feature of the face of the user 100. For example, the glasses position 112A may be measured between the eyeglass frames 114 and one or both of the pupils 106, 108. In another example, the glasses position 112B may be measured between the eyeglass frames 114 and the user's brow or forehead. In yet another example, the glasses position 112C may be measured between the eyeglass frames 114 and the medial canthus of the eye 102, 104. These glasses position 112 reference features are provided by way of example and not limitation. In additional examples, other facial features of the user 100 may be used as a reference for the glasses position(s) 112, such as a nose bridge, a corneal apex (e.g., a center of the cornea), a temple, a cheek, a lateral canthus, etc. In additional embodiments, the facial feature used to measure the glasses positions(s) 112 may be any surface of the face of the user 100.

In some examples of this disclosure, the terms “glasses,” “eyeglasses,” and “eyeglass device” may refer to any head-mounted device into which or through which a user gazes. For example, these terms may refer to prescription eyeglasses, non-prescription eyeglasses, fixed-lens eyeglasses, varifocal eyeglasses, artificial-reality glasses (e.g., augmented-reality glasses, virtual-reality glasses, mixed-reality glasses, etc.) including a near-eye display element, goggles, a virtual-reality headset for mounting a smartphone or other display device in front of the user's eyes, an ophthalmological device for measuring an optical property of the eye, etc. The eyeglasses and eyeglass devices illustrated and described in the present disclosure are not limited to the form factors shown in the drawings.

Referring to FIG. 1C, an eye relief 116 may be measured as a distance between a corneal apex 118 of the user 100 to a lens 120. The corneal apex 118 may be a center of the cornea. As the user 100 looks in different directions, the eye relief 116 may slightly change. In addition, the eye relief 116 may also change if the eyeglasses are shifted relative to the face of the user 100.

Each of the eye measurements discussed above with reference to FIGS. 1A-1C may be determined using data from ultrasound devices and systems according to the present disclosure, as will be discussed in further detail below. Such ultrasound devices and systems may also generate data for determining other measurements or derived measurements, such as focal distance, eye movement speed, eyeglass tilt, eyelid closed or open state, depth (e.g., distance) of objects in view of the user, etc.

FIG. 2 is a front view of an eyeglass device 200 illustrated over an eye 202 of a user 204, according to at least one embodiment of the present application. The eyeglass device 200 may include an eyeglass frame 206 for mounting a lens 208 and/or a display element (e.g., an NED).

An ultrasound system 210 for making eye measurements (e.g., the eye measurements discussed above with reference to FIGS. 1A-1C) may be mounted to the eyeglass frame 206 of the eyeglass device 200. The ultrasound system 210 may include at least one ultrasound transmitter T1-T3 and at least one ultrasound receiver R1-R4. The ultrasound transmitters T1-T3 may be configured and positioned to emit ultrasound signals 212 toward a face of the user 204 (e.g., toward the eye 202 and/or toward another facial feature) and the ultrasound receivers R1-R4 may be configured to detect the ultrasound signals 212 reflected from the face of the user 204. For example, the ultrasound receivers R1-R4 may be configured to generate data that can be used to determine and/or calculate a time-of-flight of the ultrasound signals 212 and/or an amplitude of the ultrasound signals 212.

The time-of-flight and/or the amplitude of the ultrasound signals 212 may be used to identify a location of a facial feature (e.g., sclera, cornea, eyelid, forehead, brow, eyelash, etc.) of the user 204. In some embodiments, the combination of time-of-flight data and amplitude data may improve a determination of the location of the facial feature compared to using only time-of-flight or amplitude data. For example, a detected ultrasound signal 212 that has a high amplitude may be more likely to be a facial feature of interest, such as a cornea, relative to a detected ultrasound signal 212 that has a low amplitude. The low amplitude ultrasound signal 212 may likely be reflected from an unintended facial feature, such as an eyelash during a blinking action.

In the example shown in FIG. 2, three ultrasound transmitters T1-T3 and four ultrasound receivers R1-R4 are illustrated. However, the present disclosure is not so limited and any number of ultrasound transmitters T1-T3 and ultrasound receivers R1-R4 may be used. In additional embodiments, there may be fewer than three ultrasound transmitters T1-T3 (e.g., a single ultrasound transmitter T1) and multiple ultrasound receivers R1-R4 (e.g., four ultrasound receivers R1-R4), multiple ultrasound transmitters T1-T3 and multiple ultrasound receivers R1-R4, multiple ultrasound transmitters T1-T3 and a single ultrasound receiver R1, or a single ultrasound transmitter T1 and a single ultrasound receiver R1.

As illustrated in FIG. 2, the ultrasound transmitters T1-T3 may be distinct and separate from the ultrasound receivers R1-R4. This configuration may enable the ultrasound signals 212 emitted by the ultrasound transmitters T1-T3 to be more easily detected at the different ultrasound receivers R1-R4, compared to ultrasound transceivers that can operate both as an emitter and a receiver. On the other hand, ultrasound receivers R1-R4 may detect a stronger signal emitted from the separate ultrasound transmitters T1-T3 and reflected from the user's eye 202 since the ultrasound receivers R1-R4 may be located to generally align with the reflected signals.

In some embodiments, the various ultrasound transmitters T1-T3 may emit unique and distinguishable ultrasound signals 212 relative to each other. For example, each of the ultrasound transmitters T1-T3 may emit an ultrasound signal 212 of a specific and different frequency. In additional examples, the ultrasound signals 212 may be modulated to have a predetermined different waveform (e.g., pulsed, square, triangular, or sawtooth). In further embodiments, any other characteristic of the ultrasound signals 212 emitted by the ultrasound transmitters T1-T3 may be unique and detectable, such that the specific source of an ultrasound signal 212 detected at the ultrasound receivers R1-R4 may be uniquely identified. In additional examples, the ultrasound transmitters T1-T3 may be activated at sequential and different times so that the ultrasound receivers R1-R4 may receive an ultrasound signal 212 from only one of the ultrasound transmitters T1-T3 during any given time period. Knowing the source of the ultrasound signal 212 may facilitate calculating a time-of-flight and/or amplitude of the ultrasound signal 212, which may improve the determination of eye measurements with the ultrasound system 210.

The ultrasound system 210 may be configured to operate at data frequencies that are higher than conventional optical sensor systems. In some examples, the ultrasound system 210 may be capable of operation at data frequencies of at least about 1000 Hz, such as 2000 Hz. Conventional optical sensor systems are generally capable of operating at about 150 Hz or less due to the increased time required to take optical images and process the images, which usually include significantly more data than ultrasound signals.

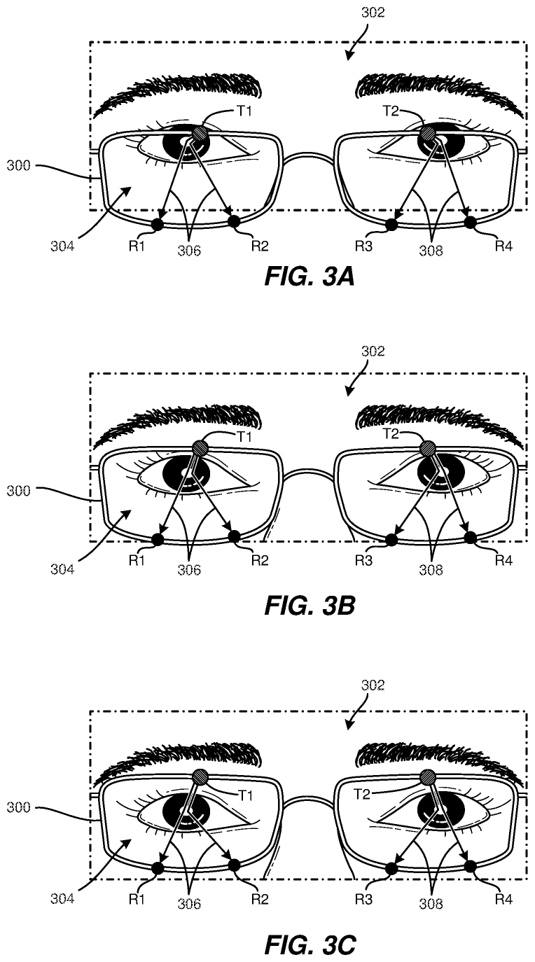

FIGS. 3A-3C are front views of an eyeglass device 300 illustrated at three different facial positions, respectively, according to at least one embodiment of the present application. FIG. 3A illustrates the eyeglass device 300 in a relatively low position on a user's face 302. FIG. 3B illustrates the eyeglass device 300 in a relatively neutral position on the user's face 302. FIG. 3C illustrates the eyeglass device 300 in a relatively high position on the user's face 302.

In some embodiments, the eyeglass device 300 may be or include an augmented-reality eyeglass device 300, which may include an NED. In this case, the position of the eyeglass device 300 on the user's face 302 may affect where on the NED an image is displayed, such as to overlay the image relative to the user's view of the real world. In additional embodiments, the eyeglass device 300 may include a varifocal lens, which may change in shape to adjust a focal distance. The focal center of the varifocal lens may be positioned at or close to a level of the user's pupil to reduce optical aberrations (e.g., blurring, distortions, etc.). Data representative of the position of the eyeglass device 300 relative to the user's eye may be useful to determine the appropriate level to locate the focal center of the varifocal lens.

In further embodiments, the eyeglass device 300 may be or include a virtual-reality HMD including a lens and an NED covering the user's view of the real world. In this case, content displayed on the NED may be adjusted (e.g., moved, refocused, etc.) based on the position of the eyeglass device 300 relative to the user's face 302. In addition, a position and/or optical property of the lens may be adjusted to reflect the position of the eyeglass device 300.

The position of the eyeglass device 300 relative to the user's face 302 may be determined using an ultrasound system 304. The ultrasound system 304 may include at least one ultrasound transmitter T1, T2 and at least one ultrasound receiver R1-R4. In FIGS. 3A-3C, a first ultrasound transmitter T1 and a first set of ultrasound receivers R1, R2 are shown over the user's right eye and a second ultrasound transmitter T2 and a second set of ultrasound receivers R3, R4 are shown over the user's left eye.

By way of example and not limitation, the first ultrasound transmitter T1 and the first set of ultrasound receivers R1, R2 may be configured to generate data to determine a position of the eyeglass device 300 relative to the user's right eye (e.g., a corneal apex of the user's right eye). To this end, the first ultrasound transmitter T1 may emit a first ultrasound signal 306, which may reflect off the user's right eye and may be detected by the first set of ultrasound receivers R1, R2. Likewise, the second ultrasound transmitter T2 and the second set of ultrasound receivers R3, R4 may be configured to generate data to determine a position of the eyeglass device 300 relative to the user's left eye (e.g., a corneal apex of the user's left eye). The second ultrasound transmitter T2 may emit a second ultrasound signal 308, which may reflect off the user's left eye and may be detected by the second set of ultrasound receivers R3, R4. In some examples, the first ultrasound signal 306 and the second ultrasound signal 308 may be distinguishable from each other (e.g., by having a different waveform, having a different frequency, being activated at different times, etc.).

The ultrasound receivers R1-R4 may be configured to sense a time-of-flight and/or an amplitude of the ultrasound signals 306, 308 reflected off the user's eyes or other facial feature. As noted above, by sensing both time-of-flight and amplitude of the ultrasound signals 306, 308, the eyeglass device 300 may more accurately and quickly determine the position of the eyeglass device 300 relative the user's face 302. As the eyeglass device 300 moves relative to the user's face 302, such as upward as shown sequentially in FIGS. 3A-3C, the ultrasound signals 306, 308 detected by the ultrasound receivers R1-R4 may be altered. For example, the time-of-flight and/or the amplitude of the ultrasound signals 306, 308 detected by one or more of the ultrasound receivers R1-R4 may change as the eyeglass device 300 moves. This change in the ultrasound signals 306, 308 may be used to identify the movement and position of the eyeglass device 300 relative to the user's face 302.

As discussed above with reference to FIG. 2, any number of ultrasound transmitters T1, T2 and any number of ultrasound receivers R1-R4 may be used in the eyeglass device 300. Moreover, in additional embodiments, the ultrasound transmitters T1, T2 and ultrasound receivers R1-R4 may be arranged on the eyeglass device 300 such that the ultrasound signals 306, 308 may be reflected from another facial feature other than the user's eyes, such as the user's brow, forehead, nose bridge, cheek, eyelid, eyelashes, medial canthus, lateral canthus, temple, etc.

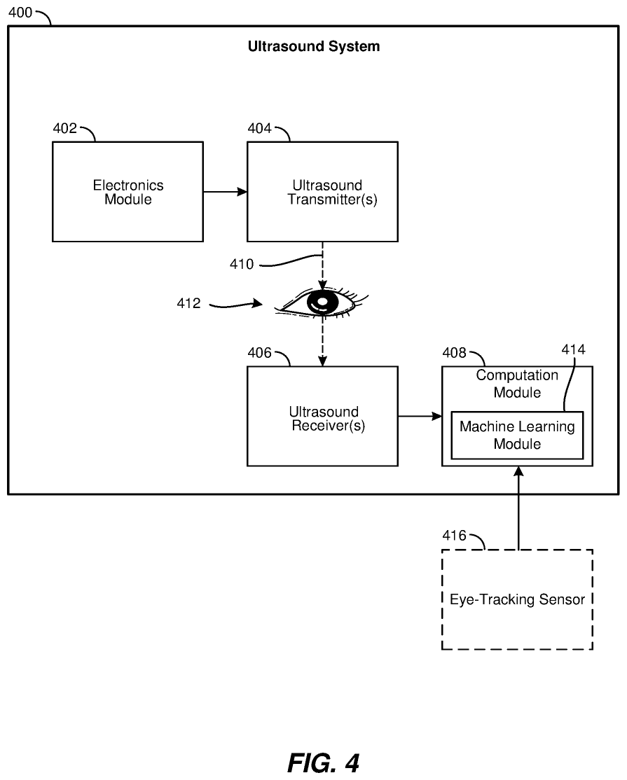

FIG. 4 is a block diagram of an ultrasound system 400 for making eye measurements, according to at least one embodiment of the present application. At least some components of the ultrasound system 400 may be mounted on an eyeglass device (e.g., on an eyeglass frame), such as any of the eyeglass devices 200, 300 discussed above.

The ultrasound system 400 may include an electronics module 402, ultrasound transmitter(s) 404, ultrasound receiver(s) 406, and a computation module 408. The electronics module 402 may be configured to generate a control signal for controlling operation of the ultrasound transmitter(s) 404. For example, the electronics module 402 may include an electronic signal generator that may generate the control signal to cause the ultrasound transmitter(s) 404 to emit a predetermined ultrasound signal 410, such as with a unique waveform for each of the ultrasound transmitters 404.

The ultrasound transmitter(s) 404 may be configured to generate ultrasound signals 410 based on the control signal generated by the electronics module 402. The ultrasound transmitter(s) 404 may convert the control signal from the electronics module 402 into the ultrasound signals 410. The ultrasound transmitter(s) 404 may be positioned and oriented to direct the ultrasound signals 410 toward a facial feature of a user, such as the user's eye 412. By way of example and not limitation, the ultrasound transmitter(s) 404 may be implemented as any of the ultrasound transmitters T1-T4 discussed above with reference to FIGS. 2 and 3A-3C.

The ultrasound receiver(s) 406 may be configured to receive and detect the ultrasound signals 410 emitted by the ultrasound transmitter(s) 404 and reflected from the facial feature of the user. As mentioned above, the ultrasound receiver(s) 406 may detect the time-of-flight and/or the amplitude of the ultrasound signals 410. The ultrasound receiver(s) 406 may convert the ultrasound signals 410 into electronic signals.

In some embodiments, the ultrasound transmitter(s) 404 and ultrasound receiver(s) 406 may be remote from each other. In other words, the ultrasound transmitter(s) 404 and the ultrasound receiver(s) 406 may not be integrated into a single ultrasound transceiver but may be separate and distinct from each other. For example, at least one of the ultrasound receivers 406 may be on an opposite side of an eyeglass frame from a corresponding ultrasound transmitter 404.

The ultrasound receiver(s) 406 may transmit data representative of the detected ultrasound signals 410 to the computation module 408. The computation module 408 may be configured to determine at least one eye measurement based on the information from the ultrasound receiver(s) 406. For example, the computation module 408 may determine the user's IPD, the position of an eyeglass device on the user's face, and/or an eye relief of the user.

The computation module 408 may determine the eye measurement(s) in a variety of ways. For example, the computation module 408 may include a machine learning module 414 configured to train a machine learning model to facilitate and improve making the eye measurement(s). Machine learning models may use any suitable system, algorithm, and/or model that may build and/or implement a mathematical model based on sample data, known as training data, in order to make predictions or decisions without being explicitly programmed to do so. Examples of machine learning models may include, without limitation, artificial neural networks, decision trees, support vector machines, regression analysis, Bayesian networks, genetic algorithms, and so forth. Machine learning algorithms that may be used to construct, implement, and/or develop machine learning models may include, without limitation, supervised learning algorithms, unsupervised learning algorithms, self-learning algorithms, feature-learning algorithms, sparse dictionary learning algorithms, anomaly detection algorithms, robot learning algorithms, association rule learning methods, and the like.

In some examples, the machine learning module 414 may train a machine learning model (e.g., a regression model) to determine the eye measurement(s) by analyzing data from the ultrasound receiver(s) 406. An initial training set of data supplied to the machine learning model may include data representative of ultrasound signals at known eye measurements. For example, if the machine learning module 414 is intended to determine glasses position, data generated by ultrasound receivers at known high, neutral, and low glasses positions may be supplied to the machine learning model. The machine learning model may include an algorithm that updates the model based on new information, such as data generated by the ultrasound receiver(s) 406 for a particular user, feedback from the user or a technician, and/or data from another sensor (e.g., an optical sensor, other ultrasound sensors, etc.). The machine learning model may be trained to ignore or discount noise data (e.g., data representative of ultrasound signals with low amplitude), which may be reflected from other facial features, such as the user's eyelashes.

In some embodiments, an optional eye-tracking sensor 416 may be included in an eyeglass device in addition to the ultrasound system 400 described above. The eye-tracking sensor 416 may include an ultrasound sensor used to periodically calibrate the ultrasound system 400 and to provide feedback to the machine learning module 414. Even with an eye-tracking sensor 416, the periodic use of the eye-tracking sensor 416 for calibrating the ultrasound system 400 may reduce power consumption and processing requirements compared to systems that rely solely on optical sensors for eye measurements. In additional embodiments, the ultrasound system 400 itself may perform eye-tracking functions without the use of the additional eye-tracking sensor 416.

FIG. 5 is a flow diagram of a method 500 for making eye measurements, according to at least one embodiment of the present application. At operation 510, at least one ultrasound transmitter may transmit an ultrasound signal toward a face of a user. Operation 510 may be performed in a variety of ways. For example, one or more ultrasound transmitters may be positioned to emit an ultrasound signal to be reflected from a corneal apex of the user's eye. In additional embodiments, the ultrasound transmitters may be positioned to emit ultrasound signals to be reflected from other facial features, as explained above.

At operation 520, at least one ultrasound receiver may receive the ultrasound signal after being reflected from the face of the user. Operation 520 may be performed in a variety of ways. For example, a plurality of ultrasound receivers may be positioned at various locations on an eyeglass frame (e.g., in locations remote from the at least one ultrasound transmitter) to receive and detect the ultrasound signal bouncing off the user's face in different directions.

At operation 530, based on information (e.g., data representative of the received ultrasound signals) from the at least one ultrasound receiver, at least one eye measurement may be determined. Operation 530 may be performed in a variety of ways. For example, a computation module employing a machine learning model may be trained to calculate a desired eye measurement (e.g., IPD, eye relief, eyeglasses position, etc.) upon receiving data from the ultrasound receiver(s). A time-of-flight of the ultrasound signals emitted by the at least one ultrasound transmitter and received by the at least one ultrasound receiver may be measured. An amplitude of the ultrasound signals may also be measured. Using both the time-of-flight and amplitude data may improve the determination of the at least one eye measurement.

Accordingly, the present disclosure includes ultrasound devices, ultrasound systems, and related methods for making various eye measurements. The ultrasound devices and systems may include at least one ultrasound transmitter and at least one ultrasound receiver that are respectively configured to emit and receive an ultrasound signal that is reflected off a facial feature (e.g., an eye, a cheek, a brow, a temple, etc.). Based on data from the at least one ultrasound receiver, a processor may be configured to determine eye measurements including IPD, eye relief, and/or an eyeglass position relative to the user's face. The disclosed concepts may enable the obtaining of eye measurements with system that is a relatively inexpensive, high-speed, accurate, low-power, and low-weight. The ultrasound systems may also be capable of processing data at a high frequency, which may be capable of sensing quick changes in the eye measurements.

Embodiments of the present disclosure may include or be implemented in conjunction with various types of artificial-reality systems. Artificial reality is a form of reality that has been adjusted in some manner before presentation to a user, which may include, for example, a virtual reality, an augmented reality, a mixed reality, a hybrid reality, or some combination and/or derivative thereof. Artificial-reality content may include completely computer-generated content or computer-generated content combined with captured (e.g., real-world) content. The artificial-reality content may include video, audio, haptic feedback, or some combination thereof, any of which may be presented in a single channel or in multiple channels (such as stereo video that produces a three-dimensional (3D) effect to the viewer). Additionally, in some embodiments, artificial reality may also be associated with applications, products, accessories, services, or some combination thereof, that are used to, for example, create content in an artificial reality and/or are otherwise used in (e.g., to perform activities in) an artificial reality.

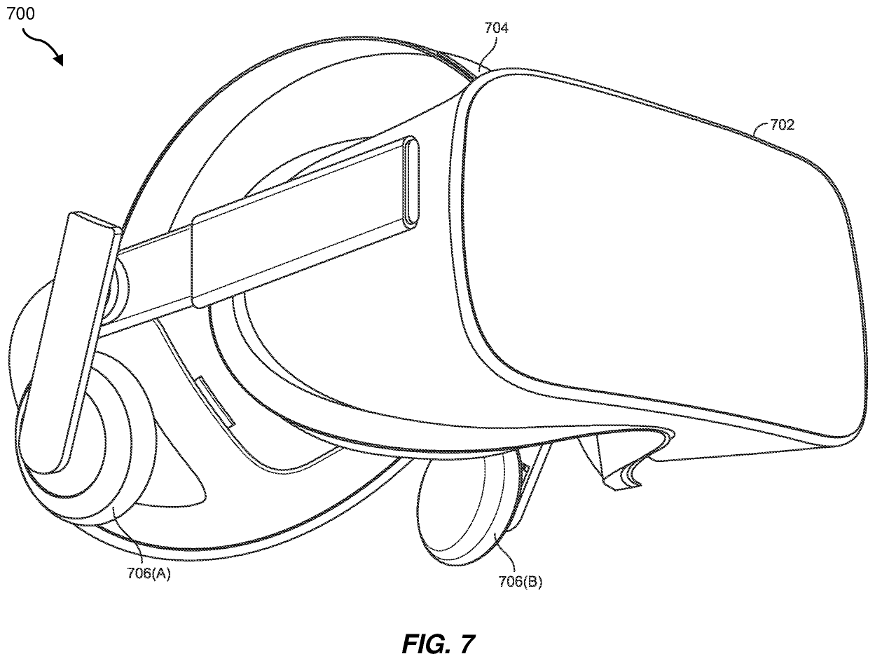

Artificial-reality systems may be implemented in a variety of different form factors and configurations. Some artificial-reality systems may be designed to work without near-eye displays (NEDs). Other artificial-reality systems may include an NED that also provides visibility into the real world (such as, e.g., augmented-reality system 600 in FIG. 6) or that visually immerses a user in an artificial reality (such as, e.g., virtual-reality system 700 in FIG. 7). While some artificial-reality devices may be self-contained systems, other artificial-reality devices may communicate and/or coordinate with external devices to provide an artificial-reality experience to a user. Examples of such external devices include handheld controllers, mobile devices, desktop computers, devices worn by a user, devices worn by one or more other users, and/or any other suitable external system.

Turning to FIG. 6, the augmented-reality system 600 may include an eyewear device 602 with a frame 610 configured to hold a left display device 615(A) and a right display device 615(B) in front of a user's eyes. The display devices 615(A) and 615(B) may act together or independently to present an image or series of images to a user. While the augmented-reality system 600 includes two displays, embodiments of this disclosure may be implemented in augmented-reality systems with a single NED or more than two NEDs.

In some embodiments, the augmented-reality system 600 may include one or more sensors, such as sensor 640. The sensor 640 may generate measurement signals in response to motion of the augmented-reality system 600 and may be located on substantially any portion of the frame 610. The sensor 640 may represent one or more of a variety of different sensing mechanisms, such as a position sensor, an inertial measurement unit (IMU), a depth camera assembly, a structured light emitter and/or detector, or any combination thereof. In some embodiments, the augmented-reality system 600 may or may not include the sensor 640 or may include more than one sensor. In embodiments in which the sensor 640 includes an IMU, the IMU may generate calibration data based on measurement signals from the sensor 640. Examples of the sensor 640 may include, without limitation, accelerometers, gyroscopes, magnetometers, other suitable types of sensors that detect motion, sensors used for error correction of the IMU, or some combination thereof.

In some examples, the augmented-reality system 600 may also include a microphone array with a plurality of acoustic transducers 620(A)-620(J), referred to collectively as acoustic transducers 620. The acoustic transducers 620 may represent transducers that detect air pressure variations induced by sound waves. Each acoustic transducer 620 may be configured to detect sound and convert the detected sound into an electronic format (e.g., an analog or digital format). The microphone array in FIG. 6 may include, for example, ten acoustic transducers: 620(A) and 620(B), which may be designed to be placed inside a corresponding ear of the user, acoustic transducers 620(C), 620(D), 620(E), 620(F), 620(G), and 620(H), which may be positioned at various locations on the frame 610, and/or acoustic transducers 620(1) and 620(J), which may be positioned on a corresponding neckband 605.

In some embodiments, one or more of the acoustic transducers 620(A)-(J) may be used as output transducers (e.g., speakers). For example, the acoustic transducers 620(A) and/or 620(B) may be earbuds or any other suitable type of headphone or speaker.

The configuration of the acoustic transducers 620 of the microphone array may vary. While the augmented-reality system 600 is shown in FIG. 6 as having ten acoustic transducers 620, the number of acoustic transducers 620 may be greater or less than ten. In some embodiments, using higher quantities of the acoustic transducers 620 may increase the amount of audio information collected and/or the sensitivity and accuracy of the audio information. In contrast, using a lower quantity of acoustic transducers 620 may decrease the computing power required by an associated controller 650 to process the collected audio information. In addition, the position of each acoustic transducer 620 of the microphone array may vary. For example, the position of an acoustic transducer 620 may include a defined position on the user, a defined coordinate on the frame 610, an orientation associated with each acoustic transducer 620, or some combination thereof.

The acoustic transducers 620(A) and 620(B) may be positioned on different parts of the user's ear, such as behind the pinna, behind the tragus, and/or within the auricle or fossa. Or, there may be additional acoustic transducers 620 on or surrounding the ear in addition to the acoustic transducers 620 inside the ear canal. Having an acoustic transducer 620 positioned next to an ear canal of a user may enable the microphone array to collect information on how sounds arrive at the ear canal. By positioning at least two of the acoustic transducers 620 on either side of a user's head (e.g., as binaural microphones), the augmented-reality device 600 may simulate binaural hearing and capture a 3D stereo sound field around about a user's head. In some embodiments, the acoustic transducers 620(A) and 620(B) may be connected to the augmented-reality system 600 via a wired connection 630, and in other embodiments the acoustic transducers 620(A) and 620(B) may be connected to the augmented-reality system 600 via a wireless connection (e.g., a BLUETOOTH connection). In still other embodiments, the acoustic transducers 620(A) and 620(B) may not be used at all in conjunction with the augmented-reality system 600.

The acoustic transducers 620 on the frame 610 may be positioned in a variety of different ways, including along the length of the temples, across the bridge, above or below the display devices 615(A) and 615(B), or some combination thereof. The acoustic transducers 620 may also be oriented such that the microphone array is able to detect sounds in a wide range of directions surrounding the user wearing the augmented-reality system 600. In some embodiments, an optimization process may be performed during manufacturing of the augmented-reality system 600 to determine relative positioning of each acoustic transducer 620 in the microphone array.

In some examples, the augmented-reality system 600 may include or be connected to an external device (e.g., a paired device), such as the neckband 605. The neckband 605 generally represents any type or form of paired device. Thus, the following discussion of the neckband 605 may also apply to various other paired devices, such as charging cases, smart watches, smart phones, wrist bands, other wearable devices, hand-held controllers, tablet computers, laptop computers, other external compute devices, etc.

As shown, the neckband 605 may be coupled to the eyewear device 602 via one or more connectors. The connectors may be wired or wireless and may include electrical and/or non-electrical (e.g., structural) components. In some cases, the eyewear device 602 and the neckband 605 may operate independently without any wired or wireless connection between them. While FIG. 6 illustrates the components of the eyewear device 602 and the neckband 605 in example locations on the eyewear device 602 and neckband 605, the components may be located elsewhere and/or distributed differently on the eyewear device 602 and/or neckband 605. In some embodiments, the components of the eyewear device 602 and neckband 605 may be located on one or more additional peripheral devices paired with the eyewear device 602, neckband 605, or some combination thereof.

Pairing external devices, such as the neckband 605, with augmented-reality eyewear devices may enable the eyewear devices to achieve the form factor of a pair of glasses while still providing sufficient battery and computation power for expanded capabilities. Some or all of the battery power, computational resources, and/or additional features of the augmented-reality system 600 may be provided by a paired device or shared between a paired device and an eyewear device, thus reducing the weight, heat profile, and form factor of the eyewear device overall while still retaining desired functionality. For example, the neckband 605 may allow components that would otherwise be included on an eyewear device to be included in the neckband 605 since users may tolerate a heavier weight load on their shoulders than they would tolerate on their heads. The neckband 605 may also have a larger surface area over which to diffuse and disperse heat to the ambient environment. Thus, the neckband 605 may allow for greater battery and computation capacity than might otherwise have been possible on a standalone eyewear device. Since weight carried in the neckband 605 may be less invasive to a user than weight carried in the eyewear device 602, a user may tolerate wearing a lighter eyewear device and carrying or wearing the paired device for greater lengths of time than a user would tolerate wearing a heavy standalone eyewear device, thereby enabling users to more fully incorporate artificial-reality environments into their day-to-day activities.

The neckband 605 may be communicatively coupled with the eyewear device 602 and/or to other devices. These other devices may provide certain functions (e.g., tracking, localizing, depth mapping, processing, storage, etc.) to the augmented-reality system 600. In the embodiment of FIG. 6, the neckband 605 may include two acoustic transducers (e.g., 620(1) and 620(J)) that are part of the microphone array (or potentially form their own microphone subarray). The neckband 605 may also include a controller 625 and a power source 635.

The acoustic transducers 620(1) and 620(J) of the neckband 605 may be configured to detect sound and convert the detected sound into an electronic format (analog or digital). In the embodiment of FIG. 6, the acoustic transducers 620(1) and 620(J) may be positioned on the neckband 605, thereby increasing the distance between the neckband acoustic transducers 620(1) and 620(J) and other acoustic transducers 620 positioned on the eyewear device 602. In some cases, increasing the distance between the acoustic transducers 620 of the microphone array may improve the accuracy of beamforming performed via the microphone array. For example, if a sound is detected by the acoustic transducers 620(C) and 620(D) and the distance between the acoustic transducers 620(C) and 620(D) is greater than, e.g., the distance between the acoustic transducers 620(D) and 620(E), the determined source location of the detected sound may be more accurate than if the sound had been detected by the acoustic transducers 620(D) and 620(E).

The controller 625 of the neckband 605 may process information generated by the sensors on the neckband 605 and/or augmented-reality system 600. For example, the controller 625 may process information from the microphone array that describes sounds detected by the microphone array. For each detected sound, the controller 625 may perform a direction-of-arrival (DOA) estimation to estimate a direction from which the detected sound arrived at the microphone array. As the microphone array detects sounds, the controller 625 may populate an audio data set with the information. In embodiments in which the augmented-reality system 600 includes an inertial measurement unit, the controller 625 may compute all inertial and spatial calculations from the IMU located on the eyewear device 602. A connector may convey information between the augmented-reality system 600 and the neckband 605 and between the augmented-reality system 600 and the controller 625. The information may be in the form of optical data, electrical data, wireless data, or any other transmittable data form. Moving the processing of information generated by the augmented-reality system 600 to the neckband 605 may reduce weight and heat in the eyewear device 602, making it more comfortable to the user.

The power source 635 in the neckband 605 may provide power to the eyewear device 602 and/or to the neckband 605. The power source 635 may include, without limitation, lithium ion batteries, lithium-polymer batteries, primary lithium batteries, alkaline batteries, or any other form of power storage. In some cases, the power source 635 may be a wired power source. Including the power source 635 on the neckband 605 instead of on the eyewear device 602 may help better distribute the weight and heat generated by the power source 635.

As noted, some artificial-reality systems may, instead of blending an artificial reality with actual reality, substantially replace one or more of a user's sensory perceptions of the real world with a virtual experience. One example of this type of system is a head-worn display system, such as the virtual-reality system 700 in FIG. 7, that mostly or completely covers a user's field of view. The virtual-reality system 700 may include a front rigid body 702 and a band 704 shaped to fit around a user's head. The virtual-reality system 700 may also include output audio transducers 706(A) and 706(B). Furthermore, while not shown in FIG. 7, the front rigid body 702 may include one or more electronic elements, including one or more electronic displays, one or more inertial measurement units (IMUS), one or more tracking emitters or detectors, and/or any other suitable device or system for creating an artificial-reality experience.

Artificial-reality systems may include a variety of types of visual feedback mechanisms. For example, display devices in the augmented-reality system 600 and/or virtual-reality system 700 may include one or more liquid crystal displays (LCDs), light emitting diode (LED) displays, microLED displays, organic LED (OLED) displays, digital light project (DLP) micro-displays, liquid crystal on silicon (LCoS) micro-displays, and/or any other suitable type of display screen. These artificial-reality systems may include a single display screen for both eyes or may provide a display screen for each eye, which may allow for additional flexibility for varifocal adjustments or for correcting a user's refractive error. Some of these artificial-reality systems may also include optical subsystems having one or more lenses (e.g., conventional concave or convex lenses, Fresnel lenses, adjustable liquid lenses, etc.) through which a user may view a display screen. These optical subsystems may serve a variety of purposes, including to collimate (e.g., make an object appear at a greater distance than its physical distance), to magnify (e.g., make an object appear larger than its actual size), and/or to relay (to, e.g., the viewer's eyes) light. These optical subsystems may be used in a non-pupil-forming architecture (such as a single lens configuration that directly collimates light but results in so-called pincushion distortion) and/or a pupil-forming architecture (such as a multi-lens configuration that produces so-called barrel distortion to nullify pincushion distortion).

In addition to or instead of using display screens, some of the artificial-reality systems described herein may include one or more projection systems. For example, display devices in the augmented-reality system 600 and/or virtual-reality system 700 may include micro-LED projectors that project light (using, e.g., a waveguide) into display devices, such as clear combiner lenses that allow ambient light to pass through. The display devices may refract the projected light toward a user's pupil and may enable a user to simultaneously view both artificial-reality content and the real world. The display devices may accomplish this using any of a variety of different optical components, including waveguide components (e.g., holographic, planar, diffractive, polarized, and/or reflective waveguide elements), light-manipulation surfaces and elements (such as diffractive, reflective, and refractive elements and gratings), coupling elements, etc. Artificial-reality systems may also be configured with any other suitable type or form of image projection system, such as retinal projectors used in virtual retina displays.

The artificial-reality systems described herein may also include various types of computer vision components and subsystems. For example, the augmented-reality system 600 and/or virtual-reality system 700 may include one or more optical sensors, such as two-dimensional (2D) or 3D cameras, structured light transmitters and detectors, time-of-flight depth sensors, single-beam or sweeping laser rangefinders, 3D LiDAR sensors, and/or any other suitable type or form of optical sensor. An artificial-reality system may process data from one or more of these sensors to identify a location of a user, to map the real world, to provide a user with context about real-world surroundings, and/or to perform a variety of other functions.

The artificial-reality systems described herein may also include one or more input and/or output audio transducers. Output audio transducers may include voice coil speakers, ribbon speakers, electrostatic speakers, piezoelectric speakers, bone conduction transducers, cartilage conduction transducers, tragus-vibration transducers, and/or any other suitable type or form of audio transducer. Similarly, input audio transducers may include condenser microphones, dynamic microphones, ribbon microphones, and/or any other type or form of input transducer. In some embodiments, a single transducer may be used for both audio input and audio output.

In some embodiments, the artificial-reality systems described herein may also include tactile (i.e., haptic) feedback systems, which may be incorporated into headwear, gloves, body suits, handheld controllers, environmental devices (e.g., chairs, floormats, etc.), and/or any other type of device or system. Haptic feedback systems may provide various types of cutaneous feedback, including vibration, force, traction, texture, and/or temperature. Haptic feedback systems may also provide various types of kinesthetic feedback, such as motion and compliance. Haptic feedback may be implemented using motors, piezoelectric actuators, fluidic systems, and/or a variety of other types of feedback mechanisms. Haptic feedback systems may be implemented independent of other artificial-reality devices, within other artificial-reality devices, and/or in conjunction with other artificial-reality devices.

By providing haptic sensations, audible content, and/or visual content, artificial-reality systems may create an entire virtual experience or enhance a user's real-world experience in a variety of contexts and environments. For instance, artificial-reality systems may assist or extend a user's perception, memory, or cognition within a particular environment. Some systems may enhance a user's interactions with other people in the real world or may enable more immersive interactions with other people in a virtual world. Artificial-reality systems may also be used for educational purposes (e.g., for teaching or training in schools, hospitals, government organizations, military organizations, business enterprises, etc.), entertainment purposes (e.g., for playing video games, listening to music, watching video content, etc.), and/or for accessibility purposes (e.g., as hearing aids, visual aids, etc.). The embodiments disclosed herein may enable or enhance a user's artificial-reality experience in one or more of these contexts and environments and/or in other contexts and environments.

In some embodiments, the systems described herein may also include an eye-tracking subsystem designed to identify and track various characteristics of a user's eye(s), such as the user's gaze direction. The phrase “eye tracking” may, in some examples, refer to a process by which the position, orientation, and/or motion of an eye is measured, detected, sensed, determined, and/or monitored. The disclosed systems may measure the position, orientation, and/or motion of an eye in a variety of different ways, including through the use of various optical-based eye-tracking techniques, ultrasound-based eye-tracking techniques, etc. An eye-tracking subsystem may be configured in a number of different ways and may include a variety of different eye-tracking hardware components or other computer-vision components. For example, an eye-tracking subsystem may include a variety of different optical sensors, such as two-dimensional (2D) or 3D cameras, time-of-flight depth sensors, single-beam or sweeping laser rangefinders, 3D LiDAR sensors, and/or any other suitable type or form of optical sensor. In this example, a processing subsystem may process data from one or more of these sensors to measure, detect, determine, and/or otherwise monitor the position, orientation, and/or motion of the user's eye(s).

The following example embodiments are also included in the present disclosure.

Example 1: An ultrasound device for making eye measurements, which may include: at least one ultrasound transmitter positioned and configured to transmit ultrasound signals toward a user's face to reflect off a facial feature of the user's face; at least one ultrasound receiver positioned and configured to receive and detect the ultrasound signals reflected off the facial feature; and at least one processor configured to: receive data from the at least one ultrasound receiver; and determine, based on the received data from the at least one ultrasound receiver, at least one of the following eye measurements: an interpupillary distance of the user; an eye relief; or a position of a head-mounted display relative to the facial feature of the user.

Example 2: The ultrasound device of Example 1, wherein the at least one ultrasound transmitter and the at least one ultrasound receiver are positioned on an eyeglass frame.

Example 3: The ultrasound device of Example 2, wherein the eyeglass frame includes an augmented-reality eyeglasses frame.

Example 4: The ultrasound device of Example 3, wherein the augmented-reality eyeglasses frame supports at least one display element configured to display visual content to the user.

Example 5: The ultrasound device of any of Examples 1 through 4, wherein the at least one processor is further configured to determine a time-of-flight of the ultrasound signals from the at least one ultrasound transmitter to the at least one ultrasound receiver and an amplitude of the reflected ultrasound signals.

Example 6: The ultrasound device of any of Examples 1 through 5, wherein the at least one processor is further configured to use machine learning to determine the at least one of the eye measurements.

Example 7: The ultrasound device of any of Examples 1 through 6, wherein the at least one ultrasound transmitter includes a plurality of ultrasound transmitters.

Example 8: The ultrasound device of any of Examples 1 through 7, wherein the at least one ultrasound receiver comprises a plurality of ultrasound receivers.

Example 9: The ultrasound device of any of Examples 1 through 8, wherein the at least one ultrasound transmitter is further configured to transmit the ultrasound signals in a predetermined waveform.

Example 10: The ultrasound device of Example 9, wherein the predetermined waveform includes at least one of: pulsed, square, triangular, or sawtooth.

Example 11: The ultrasound device of any of Examples 1 through 10, wherein the at least one ultrasound transmitter is positioned to transmit the ultrasound signals to reflect off at least one of the following facial features of the user: an eyeball; a cornea; a sclera; an eyelid; a medial canthus; a lateral canthus; eyelashes; a nose bridge; a cheek; a temple; a brow; or a forehead.

Example 12: The ultrasound device of any of Examples 1 through 11, wherein the at least one ultrasound receiver is configured to collect and transmit data to the at least one processor at a data frequency of at least 1000 Hz.

Example 13: An ultrasound system for making eye measurements, which may include: an electronics module configured to generate a control signal; at least one ultrasound transmitter in communication with the electronics module and configured to transmit ultrasound signals toward a facial feature of a user, the ultrasound signals based on the control signal generated by the electronics module; at least one ultrasound receiver configured to receive and detect the ultrasound signals after reflecting from the facial feature of the user; and a computation module in communication with the at least one ultrasound receiver and configured to determine, based on information from the at least one ultrasound receiver, at least one of the following eye measurements: an interpupillary distance of the user; an eye relief; a position of a head-mounted display relative to an eye of the user.

Example 14: The ultrasound system of Example 13, wherein the at least one ultrasound transmitter is positioned on a frame of a head-mounted display.

Example 15: The ultrasound system of Example 13 or Example 14, wherein the at least one ultrasound receiver is positioned remote from the at least one ultrasound transmitter.

Example 16: The ultrasound system of any of Examples 13 through 15, wherein the computation module comprises a machine learning module configured to determine the at least one of the eye measurements.

Example 17: The ultrasound system of Example 16, wherein the machine learning module employs a regression model to determine the at least one of the eye measurements.

Example 18: A method for making eye measurements, which may include: transmitting, with at least one ultrasound transmitter, an ultrasound signal toward a facial feature of a face of a user; receiving, with at least one ultrasound receiver, the ultrasound signals reflected from the facial feature of the face of the user; and determining, with at least one processor and based on information from the at least one ultrasound receiver, at least one of the following eye measurements: an interpupillary distance of the user; an eye relief; or a position of a head-mounted display relative to an eye of the user.

Example 19: The method of Example 18, wherein determining the at least one of the eye measurements includes: measuring a time-of-flight of the ultrasound signals from the at least one ultrasound transmitter to the at least one ultrasound receiver; and measuring an amplitude of the ultrasound signals received by the at least one ultrasound receiver.

Example 20: The method of Example 18 or Example 19, wherein: receiving, with the at least one ultrasound receiver, the ultrasound signals includes receiving the ultrasound signals with a plurality of ultrasound receivers; and determining, with the at least one processor and based on information from the at least one ultrasound receiver, the at least one of the eye measurements includes determining the at least one of the eye measurements based on information from the plurality of ultrasound receivers.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

The preceding description has been provided to enable others skilled in the art to best utilize various aspects of the example embodiments disclosed herein. This example description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

What is claimed is:

1. An ultrasound device for making eye measurements, the ultrasound device comprising:
at least one ultrasound transmitter positioned and configured to transmit ultrasound signals toward a user's face to reflect off a facial feature of the user's face;
at least one ultrasound receiver positioned and configured to receive and detect the ultrasound signals reflected off the facial feature; and
at least one processor configured to:
receive data from the at least one ultrasound receiver; and
determine, based on the received data from the at least one ultrasound receiver, at least one of the following eye measurements:
an interpupillary distance of the user;
an eye relief; or
a position of a head-mounted display relative to the facial feature of the user.

2. The ultrasound device of claim 1, wherein the at least one ultrasound transmitter and the at least one ultrasound receiver are positioned on an eyeglass frame.

3. The ultrasound device of claim 2, wherein the eyeglass frame comprises an augmented-reality eyeglasses frame.

4. The ultrasound device of claim 3, wherein the augmented-reality eyeglasses frame supports at least one display element configured to display visual content to the user.

5. The ultrasound device of claim 1, wherein the at least one processor is further configured to determine a time-of-flight of the ultrasound signals from the at least one ultrasound transmitter to the at least one ultrasound receiver and an amplitude of the reflected ultrasound signals.

6. The ultrasound device of claim 1, wherein the at least one processor is further configured to use machine learning to determine the at least one of the eye measurements.

7. The ultrasound device of claim 1, wherein the at least one ultrasound transmitter comprises a plurality of ultrasound transmitters.

8. The ultrasound device of claim 1, wherein the at least one ultrasound receiver comprises a plurality of ultrasound receivers.

9. The ultrasound device of claim 1, wherein the at least one ultrasound transmitter is further configured to transmit the ultrasound signals in a predetermined waveform.

10. The ultrasound device of claim 9, wherein the predetermined waveform comprises at least one of: pulsed, square, triangular, or sawtooth.

11. The ultrasound device of claim 1, wherein the at least one ultrasound transmitter is positioned to transmit the ultrasound signals to reflect off at least one of the following facial features of the user:
an eyeball;
a cornea;
a sclera;
an eyelid;
a medial canthus;
a lateral canthus;
eyelashes;
a nose bridge;
a cheek;
a temple;
a brow; or
a forehead.

12. The ultrasound device of claim 1, wherein the at least one ultrasound receiver is configured to collect and transmit data to the at least one processor at a data frequency of at least 1000 Hz.

13. An ultrasound system for making eye measurements, the system comprising:
an electronics module configured to generate a control signal;
at least one ultrasound transmitter in communication with the electronics module and configured to transmit ultrasound signals toward a facial feature of a user, the ultrasound signals based on the control signal generated by the electronics module;
at least one ultrasound receiver configured to receive and detect the ultrasound signals after reflecting from the facial feature of the user; and
a computation module in communication with the at least one ultrasound receiver and configured to determine, based on information from the at least one ultrasound receiver, at least one of the following eye measurements:
an interpupillary distance of the user;
an eye relief; or
a position of a head-mounted display relative to an eye of the user.

14. The ultrasound system of claim 13, wherein the at least one ultrasound transmitter is positioned on a frame of a head-mounted display.

15. The ultrasound system of claim 13, wherein the at least one ultrasound receiver is positioned remote from the at least one ultrasound transmitter.

16. The ultrasound system of claim 13, wherein the computation module comprises a machine learning module configured to determine the at least one of the eye measurements.

17. The ultrasound system of claim 16, wherein the machine learning module employs a regression model to determine the at least one of the eye measurements.

18. A method for making eye measurements, the method comprising:
transmitting, with at least one ultrasound transmitter, an ultrasound signal toward a facial feature of a face of a user;
receiving, with at least one ultrasound receiver, the ultrasound signals reflected from the facial feature of the face of the user; and
determining, with at least one processor and based on information from the at least one ultrasound receiver, at least one of the following eye measurements:
an interpupillary distance of the user;
an eye relief; or
a position of a head-mounted display relative to an eye of the user.

19. The method of claim 18, wherein determining the at least one of the eye measurements comprises:
measuring a time-of-flight of the ultrasound signals from the at least one ultrasound transmitter to the at least one ultrasound receiver; and
measuring an amplitude of the ultrasound signals received by the at least one ultrasound receiver.

20. The method of claim 18, wherein:
receiving, with the at least one ultrasound receiver, the ultrasound signals comprises receiving the ultrasound signals with a plurality of ultrasound receivers; and
determining, with the at least one processor and based on information from the at least one ultrasound receiver, the at least one of the eye measurements comprises determining the at least one of the eye measurements based on information from the plurality of ultrasound receivers.
